(19) United States
(12) Patent Application Publication
(10) Pub. No.: US 2013/0136752 A1
(43) Pub. Date: May 30, 2013

(54) Ophthalmic Formulations

(71) Applicants: Suman Sharma, Hopkinton, MA (US); Bronwyn G. Siim, Oxford (GB)

(72) Inventors: Suman Sharma, Hopkinton, MA (US); Bronwyn G. Siim, Oxford (GB)

(73) Assignee: OXIGENE, INC., South San Francisco, CA (US)

(21) Appl. No.: 13/588,392

(22) Filed: Aug. 17, 2012

Related U.S. Application Data
(63) Continuation of application No. PCT/US2011/028185, filed on Mar. 11, 2011.
(60) Provisional application No. 61/313,037, filed on Mar. 11, 2010.

(51) Int. Cl. A61K 9/00

(52) U.S. Cl. CPC A61K 9/0051
USPC 424/158.1; 514/107; 514/130

(57) ABSTRACT

The present disclosure provides novel ophthalmic formulations for ocular administration comprising a pharmaceutically effective amount of a combretastatin, from 60% to 95% w/w pre-gelatinized starch, from 1% to 10% w/w hydrophilic matrix forming polymer, and from 0.2% to 5% lubricant.

### I. FIELD OF THE INVENTION

The present invention relates to ophthalmic formulations and ocular minitablets comprising a combretastatin, pre-gelatinized starch, hydrophilic matrix forming polymer, and a lubricant.

### II. BACKGROUND

Derived from the South African tree Combreturn caffrum, combretastatins such as combretastatin A-4 (CA4), were initially identified in the 1980's as potent inhibitors of tubulin polymerization. CA4 and other combretastatins (e.g. combretastatin A-1 (CA1)) have been shown to bind a site at or near the colchicine binding site on tubulin with high affinity. In vitro studies clearly demonstrated that combretastatins are potent cytotoxic agents against a diverse spectrum of tumor cell types in culture. CA4P and CA1P, respective phosphate prodrugs of CA4 and CA1, were subsequently developed to combat problems with aqueous insolubility (see U.S. Pat. Nos. 4,996,237; 5,409,953; and 5,569,786, each of which is incorporated herein by reference). CA1P and CA4P have also been shown to cause a rapid and acute shutdown of the blood flow to tumor tissue that is separate and distinct from the anti-proliferative effects of the agents on tumor cells themselves. A number of studies have shown that combretastatins cause extensive shut-down of blood flow within tumor microvasculature, leading to secondary tumor cell death (Dark et al., Cancer Res. 57: 1829-34, (1997); Chaplin et al., Anticancer Res. 19: 189-96, (1999); Hill et al., Anticancer Res. 22(3):1453-8 (2002); Holwell et al., Anticancer Res. 22(2A):707-11, (2002). Blood flow to normal tissues is generally far less affected by CA4P and CA1P than blood flow to tumors (Tozer et al., Cancer Res. 59: 1626-34 (1999)).

The sensitivity of abnormal and immature vasculature to combretastatin provides the basis for its use in diseases outside of oncology where abnormal neovascularization significantly contributes to pathophysiology. In preclinical models of pathologic ophthalmologic neovascularization, CA4P targets vessels with anomalous structure, resulting in an acute occlusion and reduction in blood flow. Aberrant vessels induced by overexpression of VEGF or by exposure to elevated oxygen were disrupted by systemic dose levels of 3-4 mg/kg CA4P compared to 75 mg/kg required for normal immature vessels induced by burn injury. These observations suggest that the potential for different dose responses in different indications, depending on the underlying vascular structure.

Nambu and colleagues (Investigative Ophthalmology & Visual Science 44:3650-5 (2003)) evaluated the capacity of CA4P to inhibit vascular growth in two murine models of ocular neovascularization. Transgenic mice with an overexpression of vascular endothelial growth factor (rho/VEGF mice) were administered daily IP injections of vehicle, 2.2 (6.6 mg/m²), or 4.0 (12 mg/m²) mg/kg CA4P between postnatal day 7 (P7) and postnatal day 21 (P21). At P21, the mice were euthanized and histopathology and fluorescein angiography were used to quantitate choroidal neovascularization (CNV). At P21, mice treated with vehicle or 2.2 mg/kg of CA4P showed numerous neovascular lesions. In contrast, mice treated with 4.0 mg/kg of CA4P showed a significant reduction in the number of neovascular lesions and the total area of neovascularization per retina when compared to vehicle treated mice. Thus, blood-supply deprivation has been validated as an effective therapeutic approach for ophthalmological diseases in which abnormal blood-vessel pathophysiology plays a key role, e.g., the wet form of age-related macular degeneration (ARMD), the leading cause of blindness in adults over the age of 50.

Several angiogenesis-inhibiting drugs have recently been approved for treatment of wet ARMD, but require direct injection into the eye (intravitreal injection) on a regular basis and can cause side-effects. A topically-administered anti-vascular drug, such as combretastatin A4 phosphate (a prodrug of combretastatin A4), could offer significant advantages to patients with ARMD and other ophthalmological diseases in which abnormal blood-vessel pathophysiology plays a role. Thus a need exists for suitable ophthalmic formulations that are easy to use and specifically direct the active agent to the diseased tissue of the eye.

### III. SUMMARY OF THE INVENTION

One aspect of the present disclosure provides ophthalmic formulations for ocular administration comprising a pharmaceutically effective amount of a combretastatin, from 60% to 95% w/w pre-gelatinized starch, from 1% to 10% w/w hydrophilic matrix forming polymer, and from 0.2% to 5% lubricant.

Another aspect provides ocular bioadhesive tablets comprising a pharmaceutically effective amount of a combretastatin, from 60% to 95% w/w pre-gelatinized starch, from 1% to 10% w/w hydrophilic matrix forming polymer, and from 0.2% to 5% lubricant.

Yet another aspect provides methods of treating an ocular vascular disease, said method comprising administering to a mammal in need thereof an ophthalmic formulation for ocular administration comprising a pharmaceutically effective amount of a combretastatin, from 60% to 95% w/w pre-gelatinized starch, from 1% to 10% w/w hydrophilic matrix forming polymer, and from 0.2% to 5% lubricant.

### IV. BRIEF DESCRIPTION OF THE DRAWINGS

FIG. 1 illustrates tumor volume of an ocular melanoma in response to an ophthalmic formulation as described herein.

### V. DETAILED DESCRIPTION

The present disclosure provides novel ophthalmic formulations for ocular administration comprising a pharmaceutically effective amount of a combretastatin, from 60% to 95% w/w pre-gelatinized starch, from 1% to 10% w/w hydrophilic matrix forming polymer, and from 0.2% to 5% lubricant.

Combretastatins are a class of novel vascular disrupting agents that target abnormal vasculature in oncology and ophthalmologic disorders. Originally identified as naturally occurring derivatives of the South African willow tree, Combreturn caffrum, combretastatins reversibly bind tubulin at the colchicine-binding site to inhibit microtubule assembly. Without being limited by theory, it appears that combretastatins act on pathologic neovasculature by disrupting microtubule assembly leading to the collapse of the nascent endothelial cell cytoskeleton (mature endothelial cell shape is maintained by the secondary scaffolding protein actin). These newly formed or abnormal endothelial cells then change shape from flat and elongated to rounded or spherical. This endothelial cell shape alteration causes vascular occlusion in immature and abnormal blood vessels, but has no effect on normal mature blood vessels. Selectivity depends on the differentiated state as much as on the age of the endothelial call.

In certain embodiments, the combretastatin is combretastatin A4 (CA4) or combretastatin A4 phosphate (CA4P) or a pharmaceutically acceptable salt thereof. CA4P is a synthetic phosphorylated pro-drug of CA4, a naturally occurring derivative of the South African willow tree, Combreturn caffrum, which reversibly binds tubulin at the colchicine-binding site to inhibit microtubule assembly. CA4P and CA4 disrupt microtubule assembly leading to the collapse of the nascent endothelial cell cytoskeleton (mature endothelial cell shape is maintained by the secondary scaffolding protein actin). These newly formed or abnormal endothelial cells then change shape from flat and elongated to rounded or spherical. This endothelial cell shape alteration causes vascular occlusion in immature and abnormal blood vessels, but has no effect on normal mature blood vessels. Selectivity depends on the differentiated state as much as on the age of the endothelial call. For example, in tumor vasculature, mature endothelial cells are structurally abnormal and lack an actin cytoskeleton, rending them sensitive to combretastatins.

In certain embodiments, the combretastatin useful in the present formulations is a compound of Formula I:

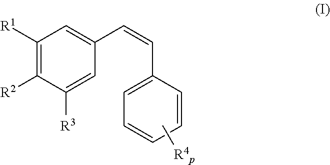

or a pharmaceutically acceptable salt thereof, wherein
each of R¹, R² and R³, independently of the others, is selected from the group consisting of hydrogen, C₁₋₆ alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR⁷ substituted with one or more of the same or R⁷ or R⁶, —B(OR⁷)₂, —B(NR⁸R⁸)₂, —(CH₂)ₘ—R⁸, —(CHR⁷)ₘ—R⁶, —S—(CH₂)ₘ—R⁶, —O—CHR⁷R⁶, —O—CR⁷(R⁶)₂, —O—(CHR⁷)ₘ—R⁶, —O— (CH₂)ₘ, —CH[(CH₂)ₘR⁶]R⁶, —S—(CHR⁷)ₘ—R⁶, —C(O)NH—(CH₂)ₘ—R⁶, —C(O)NH—(CHR⁷)ₘ—R⁶, —O—(CH₂)ₘ, —C(O)NH—(CH₂)ₘ—R⁶, —S—(CH₂)ₘ, —C(O)NH—(CH₂)ₘ—R⁶, —O—(CHR⁷)ₘ, —C(O)NH—(CHR⁷)ₘ—R⁶, —S—(CHR⁷)ₘ, —C(O)NH—(CHR⁷)ₘ—R⁶, —NH—(CH₂)ₘ—R⁶, —NH—(CHR⁷)ₘ—R⁶, —NH[(CH₂)ₘ—R⁶], —N[(CH₂)ₘ—R⁶]₂, —NH—C(O)—NH—(CH₂)ₘ—R⁶, —NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₆₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate
each R⁶ is a suitable group independently selected from the group consisting of ═O, —OR⁷, C₁₋₃ haloalkyloxy, —OCF₃, ═S, —SR⁷, ═NR⁷, ═NOR⁷, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(NOH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NHC(O)]ₙOR⁷, —[NR⁷C(O)]ₙOR⁷, —[NHC(O)]ₙNR⁸R⁸, —[NR⁷C(O)]ₙNR⁸R⁸, —[NHC(NE)]ₙNR⁸R⁸ and —[NR⁷C(NR⁷)]ₙNR⁸R⁸;
each R⁷ is independently selected from the group consisting of hydrogen, C₁₋₆ alkyl, C₃₋₈ cycloalkyl, C₄₋₁₁ cycloalkylalkyl, C₆₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R⁷ or suitable R⁶ groups;
each m independently is an integer from 1 to 3;
each n independently is an integer from 0 to 3;
p is an integer from 1 to 5, and
   wherein two adjacent R⁴ groups and their intervening atoms can be bonded to form a 5-8 membered ring fused to the central phenyl group.

In another embodiment, the combretastatin is a phosphate prodrug of a combretastatin, or a pharmaceutically acceptable salt thereof. An exemplary phosphate prodrug is a compound of Formula II:

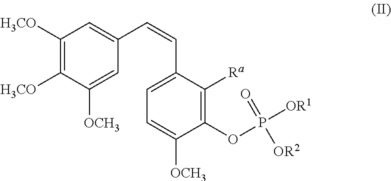

wherein
R^{a} is H or OP(O)(OR³)OR⁴; and
OR¹, OR², OR³ and OR⁴ are each, independently, OH, —O⁻QH⁺ or —O⁻M⁺, wherein M⁺ is a monovalent or divalent metal cation and Q is, independently:
   a) an amino acid containing at least two nitrogen atoms where one of the nitrogen atoms, together with a proton, forms a quaternary ammonium cation QH⁺; or
   b) an organic amine containing at least one nitrogen atom which, together with a proton, forms a quaternary ammonium cation, Q⁺.

In one embodiment of Formula II, R^{a} is H, one of OR¹ and OR² is hydroxyl, and the other is —O⁻QH⁺ where Q is tris(hydroxymethyl)amino methane (“TRIS” or “tromethamine”).

In another embodiment of Formula II, R^{a} is H or OP(O)(OR³)OR⁴, and R¹, R², R³ and R⁴ are each, independently, an aliphatic organic amine, alkali metals, transition metals, heteroarylene, heterocyclyl, nucleoside, nucleotide, alkaloid, amino sugar, amino nitrile, or nitrogenous antibiotic.

In another embodiment of Formula II, R¹, R², R³ and R⁴ are each, independently, Na, tromethamine, histidine, ethanolamine, diethanolamine, ethylenediamine, diethylamine, triethanolamine, glucamine, N-methylglucamine, ethylenediamine, 2-(4-imidazolyl)-ethylamine, choline, or hydrabamine.

In another embodiment, Formula II is represented by a compound of Formula III:

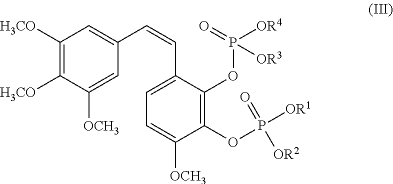

wherein
OR¹, OR², OR³ and OR⁴ are each, independently, OH, —O⁻QH⁺ or —O⁻M⁺, wherein M⁺ is a monovalent or divalent metal cation, and Q is, independently:
   a) an amino acid containing at least two nitrogen atoms where one of the nitrogen atoms, together with a proton, forms a quaternary ammonium cation QH⁺; or
   b) an organic containing at least one nitrogen atom which, together with a proton, forms a quaternary ammonium cation, QH⁺.

In one embodiment of Formula III, at least one of OR¹, OR², OR³ and OR⁴ is hydroxyl, and at least one of OR¹, OR², OR³ and OR⁴ is —O⁻QH⁺, where Q is tromethamine.

In some embodiments, the combretastatin may be combretastatin A-4 or an analog, prodrug or derivative thereof. In some implementations, the combretastatin is combretastatin A-4 phosphate or a pharmaceutically acceptable salt thereof. In a preferred implementation, the combretastatin is a tromethamine salt of combretastatin A-4 phosphate.

An “effective amount,” which is also referred to herein as a “therapeutically effective amount,” of a combretastatin for administration as described herein is that amount of the combretastatin that provides the therapeutic effect sought when administered to the subject, including but not limited to a human subject. The achieving of different therapeutic effects may require different effective amounts of combretastatin. For example, the therapeutically effective amount of a combretastatin used for preventing a disease or condition may be different from the therapeutically effective amount used for treating, inhibiting, delaying the onset of, or causing the regression of the disease or condition. In addition, the therapeutically effective amount may depend on the age, weight, and other health conditions of the subject as is well know to those versed in the disease or condition being addressed. Further, the therapeutically effective amount can depend upon the route of administration. Thus, the therapeutically effective amount may not be the same in every subject to which the combretastatin is administered.

An effective amount of a combretastatin for treating, preventing, inhibiting, delaying the onset of, or causing the regression of an ophthalmological disease in which abnormal blood-vessel pathophysiology plays a key role is also referred to herein as the amount of combretastatin effective to treat, prevent, inhibit, delay the onset of, or cause the regression of the ophthalmological disease.

To determine whether a level of combretastatin is a “therapeutically effective amount” to treat, prevent, inhibit, delay on set of, or cause the regression of an ophthalmological disease in which abnormal blood-vessel pathophysiology plays a key role, formulations may be administered in animal models for the ophthalmological disease, and the effects may be observed. In addition, dose ranging human clinical trials may be conducted to determine the therapeutically effective amount of a combretastatin.

With mammals, including humans, the effective amounts can be determined by standard method and administered on the basis of body surface area. The interrelationship of dosages varies for animals of various sizes and species, and for humans (based on mg/m² of body surface) is described by E. J. Freireich et al., Cancer Chemother. Rep., 50(4) δ 219 (1966). Body surface area may be approximately determined from the height and weight of an individual (see, e.g., Scientific Tables, Geigy Pharmaceuticals, Ardsley, N.Y. pp. 537-538 (1970)). A suitable dose range is from 1 to 1000 mg of equivalent per m² body surface area of a combretastatin, for instance from 50 to 500 mg/m².

The ophthalmic formulation described herein may also include an additional active agent. In some embodiments, the ophthalmic formulation includes verteporfin (Visudyne™) In some embodiments the ophthalmic formulation includes an inhibitor of Vascular Endothelial Growth Factor (VEGF). In some embodiments the ophthalmic formulation includes an inhibitor of Vascular Endothelial Growth Factor-A (VEGF-A). In some embodiments, the inhibitor of VEGF is a VEGF trap molecule including, without limitation, aflibercept. In some embodiments the inhibitor of VEGF is an antibody or fragment thereof directed to VEGF. In some embodiments the antibody or fragment thereof directed to VEGF is bevacizumab (i.e., Avastin™). In some embodiments the antibody or fragment thereof directed to VEGF is ranibizumab (i.e., Lucentis™).

Additional active agents that may be included in the ophthalmic formulation include analgesics, anesthetics, or anti-inflammatory agents. In some embodiments, active agents that may be used in the ophthalmic formulations are anti-inflammatory agents (such as hydrocortisone, dexamethasone, fluocinolone, prednisone, prednisolone, methylprednisolone, fluorometholone, betamethasone and triamcinolone), antihistamines (such as cetirizine hydrochloride, clemastine fumarate, promethazine, loratidine, desloratadine, diphenhydramine hydrochloride, fexofenadine hydrochloride, acrivastine, astemizole, azelastine, ebastine, epinastine, and mizolastine), antibiotics (such as tetracycline, chlortetracycline, bacitracin, neomycin, polymyxin, gramicidin, cephalexin, oxytetracycline, chloramphenicol, rifampicin, ciprofloxacin, levofloxacin, Gatifloxacin, moxifloxacin, aminosides, gentamycin, erythromycin, penicillin, quinolone, ceftazidime, vancomycin, imipeneme, sulfonamides, sulfadiazine, sulfacetamide, sulfamethizole, sulfisoxazole, nitrofurazone and sodium propionate), antifungals (such as amphotericin B, fluconazole, ketoconazole and miconazole), anti-allergics (such as sodium cromoglycate, antazoline, methapyriline, chlorpheniramine, cetirizine, pyrilamine and prophenpyridamine), antiprotozoal agents, antiviral agents, antifungal agents, anti-infective agents, antimetabolites, and antiangiogenic agents.

In addition, the ophthalmic formulations of the present invention can, optionally, include a diagnostic aid, particularly fluorescent probes, such as fluorescein or rose bengal. In certain embodiments, fluorescent probes have excitation and emission wavelengths in the red and near infrared spectrum in the range 550-1300 or 400-1300 nm or about 440 and about 1100 nm, between about 550 and about 800 nm, between about 600 and about 900 nm. Use of this portion of the electromagnetic spectrum maximizes tissue penetration and minimizes absorption by physiologically abundant absorbers such as hemoglobin (<650 nm) and water (>1200 nm). In particular, fluorophores such as certain carbocyanine or polymethine fluorescent fluorochromes or dyes can be used to construct optical imaging agents. Exemplary fluorochromes for probes useful in the present formulations include, inter alia: Cy5.5, Cy5, Cy7.5 and Cy7 (GE Healthcare); AlexaFluor660, AlexaFluor680, AlexaFluor790, and AlexaFluor750 (Invitrogen); VivoTag™ 680, VivoTag™-5680, VivoTag™-5750 (VIsEN Medical); Dy677, Dy682, Dy752 and Dy780 (Dyomics); DyLight® 547, and/or DyLight® 647 (Pierce); HiLyte Fluor™ 647, HiLyte Fluor™ 680, and HiLyte Fluor™ 750 (AnaSpec); IRDye® 800CW, IRDye® 800RS, and IRDye® 700DX (Li-Cor.®); ADS780WS, ADS830WS, and ADS832WS (American Dye Source); XenoLight CF™ 680, XenoLight CF™ 750, XenoLight CF™ 770, and XenoLight DiR (Caliper Life Sciences); and Kodak X-SIGHT® 650, Kodak X-SIGHT 691, Kodak X-SIGHT 751 (Carestream Health).

The amount of hydrophilic matrix forming polymer in the ophthalmic formulations according to the present invention in general ranges from 1% to 10% (w/w), and most preferably is about 5% (w/w). These polymers will be non-toxic, that is safe for human consumption when administered topically to the eye. Examples of hydrophilic matrix forming polymers are polyacrylic acid (carbomer), hydroxyethyl cellulose, hydroxypropyl cellulose, hydroxypropyl methylcellulose, carboxymethylcellulose, polyvinyl alcohol (PVA), alginic acids and salts and/or mixtures thereof. These polymers, and the group of polymers of like nature, provide two benefits: i) the matrix they form with the active agent effects a sustained release preparation, and ii) when exposed to aqueous media, the polymers demonstrate a wetting, swelling and/or adhesive behavior. Polyacrylic acid and in particular carbomer 974P (as known as carbomer 934P) is useful in ensuring that the dosage forms prepared from the bioadhesive compositions have a regular and prolonged release pattern of the active ingredient. Therefore it is the preferred hydrophilic matrix forming polymer in the bioadhesive compositions according to the present invention. Other polymers particularly suitable for use in the present compositions include Carbomer 940, Carbomer 941, Carbomer 971P, Carbomer 980, Carbomer 1342, Carbomer ETD, Carbomer 71G, polycarbophil and calcium polycarbophil.

The amount of pregelatinized starch in the ophthalmic formulations of the present invention typically range from about 60% to about 95% (w/w). Pregelatinized starches are cheap products. They are manufactured by precooking and drying starches, and are widely used in the food industry in order to give viscous pastes after reconstitution in water. They are mainly used by users who do not have the facilities for cooking starch. Besides the food industry they are also used in the preparation of oil-well drilling mud and in foundry cores for metal casting.

Pregelatinization is easily obtained by:
spray-drying: these products consist of distorted hollow spheres, usually with an air cell enclosed at the center. They are made by first cooking the starch in water and then by spraying the hot paste into a drying chamber or tower:
roll-dried: particles appear as transparent, flat irregular platelets. In general these products are simultaneously cooked and dried on heated rolls, using either a closely set pair of squeeze rolls or a single roll with a closely set doctor blade. In either case, a paper thin flake, which is then ground to the desired mesh size, is obtained;
extruded or drum-dried: individual particles from either process are much thicker and more irregular in dimensions than roll-dried products. Drum-drying is similar to roll-drying except that a thicker coating of starch paste is applied to the heated rolls, and the dried product is the ground to the desired particle size. In the extruded process, moistened starch is forced through a super heated chamber under very high shear, then “exploded” and simultaneously dried by venting at atmospheric pressure.
Pregelatiniged starch (DDWM) is used for its direct compression property

Preferably, the lubricant is present in an amount between 0.2 and 5.0 percent by weight relative to the weight of the ophthalmic formulation. Any lubricant that performs the function of preventing powder from sticking to the tooling may be used. Preferred lubricants include but are not limited to stearic acid, glyceryl behenate, magnesium stearate, calcium stearate, light mineral oil, polyethylene glycol, sodium stearyl fumarate, and hydrogenated vegetable oil. A preferred lubricant, sodium stearyl fumarate, typically is more hydrophilic than traditional lubricants, less sensitive to blending and relatively inert. This results in tablets with improved disintegration and dissolution, harder tablets and better drug stability.

In some embodiments, the ophthalmic formulations described herein are administered by topical administration. In some embodiments, the ophthalmic formulation is applied topically to the eye any of 1, 2, 3, 4, or 5 times per day. In some embodiments, the ophthalmic formulation is applied topically to the eye about once or less any of about every 1, 2, 3, 4, 5, 6, 7, 10, 14, 21, or 28 day(s). In some embodiments, the ophthalmic formulation is applied topically to the eye about once or less a day. In some embodiments, the ophthalmic formulation is applied topically to the eye about once or less every 5 days. In some embodiments, the ophthalmic formulation is applied topically to the eye about once or less of every 10 days.

In some embodiments, a total amount of combretastatin less than about 5 mg is administered. In some embodiments, a total amount of combretastatin less than about 5.0 mg is administered. In some embodiments, a total amount of combretastatin less than about 4.5 mg is administered. In some embodiments, a total amount of combretastatin less than about 4.0 mg is administered. In some embodiments, a total amount of combretastatin less than about 3.5 mg is administered. In some embodiments, a total amount of combretastatin less than about 3.0 mg is administered. In some embodiments, a total amount of combretastatin less than about 2.5 mg is administered. In some embodiments, a total amount of combretastatin less than about 2 mg is administered. In some embodiments, a total amount of combretastatin less than about 1.2 mg is administered. In some embodiments, a total amount of combretastatin less than about 1.0 mg is administered. In some embodiments, a total amount of combretastatin less than about 0.8 mg is administered. In some embodiments, a total amount of combretastatin less than about 0.6 mg is administered. In some embodiments, a total amount of combretastatin less than about 0.4 mg is administered. In some embodiments, a total amount of combretastatin administered is any of between about 20 μg and about 4000 μg, between about 10 μg and about 2000 μg, between about 10 μg and 1750 μg, between about 1500 μg and 1000 μg, or between about 10 μg and 1000 μg.

The ophthalmic formulations and ocular tablets described herein may be used to deliver amounts of the combretastatin effective for treating, preventing, inhibiting, delaying on set of, or causing the regression of an ophthalmological disease in which abnormal blood-vessel pathophysiology plays a key role. In some embodiments the formulations described herein deliver the combretastatin and one or more additional active agents over an extended period of time.

Ophthalmological diseases treatable by the non-systemic administration of a combretastatin in accordance with the present invention include non-malignant vascular proliferative diseases characterized by corneal, retinal, or choroidal neovascularization, as well as malignant vascular proliferative diseases such as ocular tumors and cancers. Examples of ophthalmological diseases susceptible to treatment with the formulations of the present invention include, but are limited to, proliferative retinopathies, choroidal neovascularization (CNV), macular degeneration, diabetic and other ischemia-related retinopathies, diabetic macular edema, cystoids macular edema, pathological myopia, von Hippl-Landau disease, pathological choroidal vasculopathy (PCV), histoplasmosis of the eye, Central Retinal Vein Occlusion (CRVO), corneal neovascularization, retinal neovascularization, neovascular glaucoma, retinopathy of prematurity, vascularization of the cornea secondary to injury, retinitis pigmentosa (RP), uveal melanoma, retinoblastoma, choroidal melanoma, intraocular melanoma, and primary ocular lymphoma.

In a further aspect, provided herein are kits comprising one or more unit dose forms as described herein. In some embodiments, the kit comprises one or more of packaging and instructions for use to treat one or more diseases or conditions. In some embodiments, the kit comprises a diluent which is not in physical contact with the formulation or pharmaceutical formulation. In some embodiments, the kit comprises any of one or more unit dose forms described herein in one or more sealed vessels. In some embodiments, the kit comprises any of one or more sterile unit dose forms.

In some embodiments, the kit comprises a container for the ophthalmic formulation or ocular tablet of the present invention. Suitable containers include, for example, a bottle, a box, a blister card, a foil packet, or a combination thereof. Optionally, the kit also contains directions for properly administering the ophthalmic formulations or tablets. The kits can also be designed in a manner such that they are tamper resistant or designed to indicate if tampering has occurred. Optionally, the kit of the present invention can contain the ophthalmic formulation or tablet of the present invention in combination with other pharmaceutical compositions. In some embodiments, the ophthalmic formulation or tablet is an individual dosage unit.

Optionally associated with the container(s) in the kits of the present invention can be a notice or printed instructions. Such printed instructions can be in a form prescribed by a governmental agency regulating the manufacture, use, or sale of pharmaceuticals or biological products, which notice reflects approval by the agency of the manufacture, use, or sale for human administration to treat a condition that could be treated by combretastatin therapy. In some embodiments, the kit further comprises printed matter, which, e.g., provides information on the use of the ophthalmic formulation or ocular tablet to treat a condition or disease or a pre-recorded media device which, e.g., provides information on the use of the ophthalmic formulation or tablet to treat a condition or disease, or a planner.

The kit can also include a container for storing the other components of the kit. The container can be, for example, a bag, box, envelope or any other container that would be suitable for use in the present invention. Preferably, the container is large enough to accommodate each component and/or any administrative devices that may be accompany the ophthalmic formulations or tablets of the present invention.

### VI. EXAMPLES

### A. Example 1

### Manufacture of Combretastatin A4 Phosphate Ocular Tablets

TABLE 1
Tablet Compositions

| | Amount per tablet [mg (% w/w)] | | | | | |
|---|---|---|---|---|---|---|
| tablet strength | 2.0 mg | 1.0 mg | 0.5 mg | 0.3 mg | 0.1 mg | 0.03 mg |
| CA4P.tris* | 2.66 (33.2) | 1.33 (16.6) | 0.66 (8.3) | 0.39 (4.9) | 0.13 (1.6) | 0.039 (0.49) |
| Drum dried waxy | 4.9 (60.8) | 6.2 (77.4) | 6.9 (85.7) | 7.1 (89.1) | 7.4 (92.4) | 7.5 (94.51) |
| maize starch | | | | | | |
| Carbopol 974P | 0.40 (5.0) | 0.40 (5.0) | 0.40 (5.0) | 0.40 (5.0) | 0.40 (5.0) | 0.40 (5.0) |
| Sodium stearyl | 0.08 (1.0) | 0.08 (1.0) | 0.08 (1.0) | 0.08 (1.0) | 0.08 (1.0) | 0.08 (1.0) |
| fumarate | | | | | | |
| Tablet Weight | 8.00 (100) | 8.00 (100) | 8.00 (100) | 8.00 (100) | 8.00 (100) | 8.00 (100) |

*1.316 g of combretastatin A4 phosphate tromethamine (CA4P.tris) is equivalent to 1 g of free acid combretastatin A4 phosphate.

The drug was sieved through mesh #40 prior to dispensing. Carbopol 974P and drum dried waxy maize starch were dispensed and sifted through mesh #40 (ASTM) sieve. The sieved material was blended using a geometric mixing technique in a polybag for 5 minutes to get a blend with acceptable content uniformity. The powder blend was lubricated with part quantity (0.04 mg per tablet) of sifted (#40 passed) sodium stearyl fumarate for 2 minutes and the lubricated blend was slugged to form compacts. These compacts were deslugged and sifted through mesh #40 (ASTM) sieve. The remaining quantity of sifted (#40 passed) sodium stearyl fumarate (0.04 mg per tablet) was added to the sieved granules and lubricated for additional 2 minutes in a polybag.

The lubricated granules were compressed using a 2.5 mm multi-tip punch set at an average weight of 8 mg. The physical properties of the tablets were evaluated for their thickness, hardness, average weight, and also the release profile in a medium comprising of phosphate buffer (pH—7.4). The physical properties of the tablets are summarized in Table 2.

TABLE 2
physical properties of tablet

| | 0.3 mg | 0.1 mg | 0.03 mg |
|---|---|---|---|
| Thickness (mm) | 1.5-1.6 | 1.5-2.0 | 1.5-2.0 |
| Hardness (kp) | 1.0-1.5 | 0.5-1.5 | 0.5-1.5 |
| Average Weight (mg) | 8.05 | 7.9 | 7.9 |

The oscillating water bath method was used for dissolution testing as it simulates the conditions in the ocular region. The dissolution was measured in phosphate buffer (pH 7.4), at an oscillating frequency of 25, 32° C., in a media volume of 8 mL with a 1 mL replacement volume. The results from dissolution studies using oscillating water bath is compiled in Table 3.

TABLE 3
Dissolution profile of tablets

| | % Drug Dissolved | | |
|---|---|---|---|
| Time (hrs) | 0.3 mg | 0.1 mg | 0.03 mg |
| 0.5 | 12 | 16 | 34 |
| 1.0 | 36 | 38 | 43 |
| 2.0 | 62 | 59 | 64 |
| 4.0 | 84 | 80 | 81 |
| 6.0 | 94 | 86 | 86 |
| 8.0 | 97 | 85 | 86 |

### B. Example 2

### Ocular Tissue Penetration

One hundred and eight (108) pigmented rabbits from the Fauve de Bourgogne strain were randomly divided into twenty-seven (27) groups of four (4) animals each. Table 4 below summarizes the allocation of animals in treatment groups:

TABLE 4
Study Design

| Group | Formulation | Administration | Time (h) |
|---|---|---|---|
| 1 | 10 mg/mL* CA4P-tris | 30 μl instillation into | 0.5 |
| 2 | ophthalmic solution | both eyes | 1 |
| 3 | | | 2 |
| 4 | | | 4 |
| 5 | | | 8 |
| 6 | | | 12 |
| 7 | | | 24 |
| 8 | placebo ophthalmic | | 0.5 |
| 9 | solution | | 2 |
| 10 | CA4P-tris 0.3 mg* | insertion into fornix of | 0.5 |
| 11 | minitablet | both eyes | 1 |
| 12 | | | 2 |
| 13 | | | 4 |
| 14 | | | 8 |
| 15 | | | 12 |
| 16 | | | 24 |
| 17 | placebo minitablet | | 0.5 |
| 18 | | | 2 |
| 19 | CA4P-tris ophthalmic | 10 mg/kg intraperitoneal | 0.5 |
| 20 | solution | injection | 1 |
| 21 | | | 2 |
| 22 | | | 4 |
| 23 | | | 8 |
| 24 | | | 12 |
| 25 | | | 24 |
| 26 | placebo ophthalmic | 1 mL/kg intraperitoneal | 0.5 |
| 27 | solution | injection | 2 |

*Combretastatin amount based upon free acid equivalent

At the time-points listed in Table 1, animals were anesthetized 10 minutes before euthanasia using an intramuscular injection of xylazine and ketamine. Whole blood (10 mL) was sampled into K₂EDTA tubes for plasma preparation. After euthanasia by a cardiac injection of overdose pentobarbital, cornea (C), aqueous humor (AH), irisciliary body (ICB), vitreous (V) and choroid/retina (CHR) were sampled from each eye, snap-frozen separately in liquid nitrogen and stored at −80° C. until assay. “Retina choroid” and “retina choroid tissues,” as used herein, are synonymous and refer to the combined retina and choroid tissues of the eye.

Before assay, CA4 and CA4P and internal standard (added after defrosting the sample and just before extraction) were extracted from the C, AH, ICB, V, CHR and plasma. Then, CA4 and CA4P concentrations were determined by RRLC-MS/MS. From the measured concentrations, maximum concentration (Cₘₐₓ), half-life (T_{1/2}), time at which maximum concentration was measured (Tₘₐₓ), and area under the curve (AUC) were calculated according standard methods. Table 5 provides a summary of Cₘₐₓ, T_{1/2}, Tₘₐₓ and AUC for the prodrug, CA4P, in each ocular structure and plasma. Table 6 provides a summary of Cₘₐₓ, T_{1/2}, Tₘₐₓ and AUC for the active drug, CA4, in each ocular structure and plasma.

TABLE 5
Combretastatin A4 Phosphate Values

| | vitreous | | | iris/ciliary body | | | choroid/retina | | | plasma | | |
|---|---|---|---|---|---|---|---|---|---|---|---|---|
| | drop | tab | i.p. | drop | tab | i.p. | drop | tab | i.p. | drop | tab | i.p. |
| Cₘₐₓ | 4.0 | 115.1 | 2.1 | 22.3 | 614.2 | 17.2 | 71.3 | 805.7 | 5.6 | 0.6 | 5.2 | 3151.1 |
| Tₘₐₓ | 0.5 | 0.5 | 1.0 | 0.5 | 2.0 | 0.5 | 2.0 | 2.0 | 0.5 | 0.5 | 2.0 | 0.5 |
| T_{1/2} | 30.34 | 1.65 | 0.37 | 0.67 | NA | 0.7 | 101.66 | 0.95 | 2.36 | NA | NA | 0.81 |
| AUC | 39.7 | 57.8 | 2.3 | 24.5 | 1262.4 | 19.9 | 478.7 | 1531.0 | 5.0 | 0.2 | 9.9 | 3764.9 |

TABLE 6
Combretastatin A4 Values

| | vitreous | | | iris/ciliary body | | | choroid/retina | | | plasma | | |
|---|---|---|---|---|---|---|---|---|---|---|---|---|
| | drop | tab | i.p. | drop | tab | i.p. | drop | tab | i.p. | drop | tab | i.p. |
| Cₘₐₓ | 225.6 | 160.0 | 2.0 | 393.2 | 815.7 | 98.0 | 92.5 | 568.0 | 310.3 | 5.7 | 5.9 | 419.9 |
| Tₘₐₓ | 0.5 | 2.0 | 0.5 | 0.5 | 2.0 | 1.0 | 0.5 | 2.9 | 1.0 | 0.5 | 2.0 | 0.5 |
| T_{1/2} | 0.39 | 0.77 | 0.96 | 0.54 | 0.44 | 1.57 | 9.17 | 0.43 | 1.46 | 0.22 | NA | 0.73 |
| AUC | 134.8 | 349.0 | 2.6 | 344.3 | 1686.7 | 252.8 | 238.0 | 1095.5 | 766.1 | 2.3 | 11.9 | 589.9 |

Following a single instillation (10 mg/mL, 30 μL in both eyes), minitablet administration (0.3 mg/insert in both eyes) or intraperitoneal injection (10 mg/kg), the Cₘₐₓ of CA4 and CA4P in each ocular structure was higher after minitablet administration than after instillation or intraperitoneal injection, except for the CA4 in aqueous humor (Cmax instillation>Cmax minitablet>Cmax intraperitoneal injection). The site specific exposure (AUC in ocular structure divided by AUC in plasma) to the active combretastatin was significantly higher when using the minitablet formulation as compared to i.p. injection (Table 7).

TABLE 7
Site specific exposure to CA4P and CA4

| | AUC_{0.5h-24h} (ng/g of tissue or ng/mL plasma × hour) | | |
|---|---|---|---|
| | Eye Drop | Mini Tablet | IP injection |
| Iris Ciliary Body | 369 | 2949 | 273 |
| Aqueous Humor | 175 | 407 | 4.9 |
| Plasma | 2.5 | 21.8 | 4355 |
| Retina/Choroid | 717 | 2627 | 771 |
| Ratio R/C to | 287 | 121 | 0.18 |
| plasma | | | |

### C. Example 3

### Treatment of Ocular Melanoma

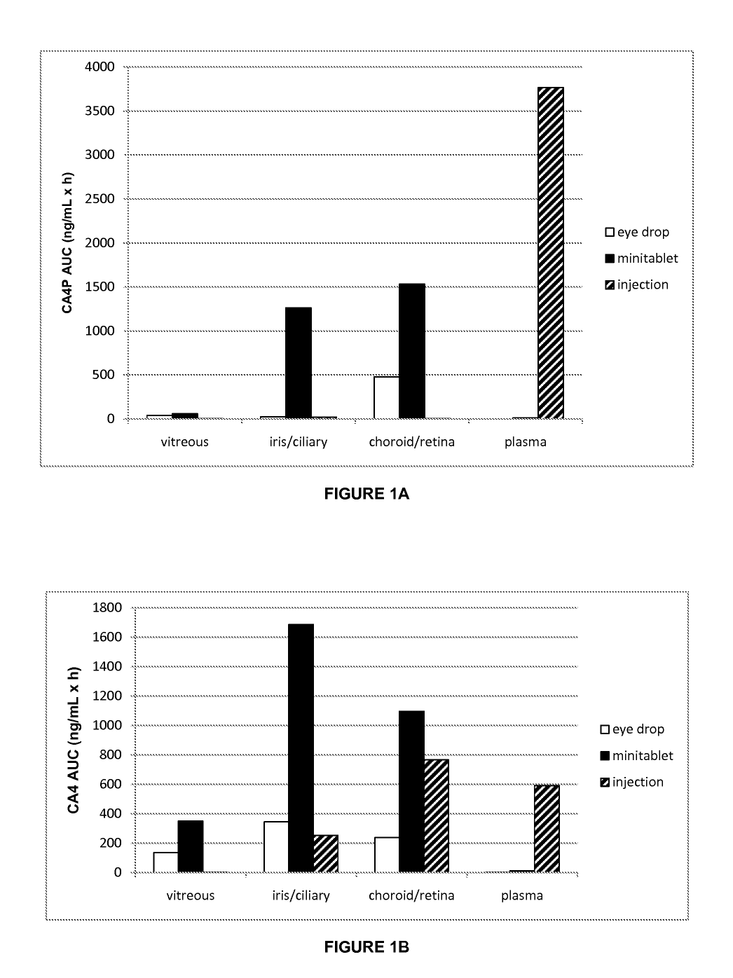

The surprisingly high penetration of ophthalmic formulations of the invention is confirmed by the efficacy observed in a rat melanoma study in which rats had spheroids grown from C918 human choroidal melanoma cells implanted into the suprachoroidal space of the right eye. Treatment began the day after implantation. There were two treatment groups plus a control group. Two groups of rats received either a 30 μl drop of a 1% CA4P solution or vehicle once a day five days a week (Monday-Friday). The third group had a minitablet placed in the right eye once a day. Every seven days tumor volume was quantified noninvasively using high-frequency ultrasound, and the rats were weighed. Rats were followed until the tumor grew too large (volume>50 mm3). Rats greater than 123 days of age at implantation were subsequently excluded from the study due to problems of weight loss in all arms of the study including the control arm. FIG. 1 provides a summary of tumor volumes at various time points after implantation of the minitablet as compared to the control.

We claim:

1. An ophthalmic formulation for ocular administration comprising
(a) a pharmaceutically effective amount of a combretastatin;
(b) from 60% to 95% w/w pre-gelatinized starch;
(c) from 1% to 10% w/w hydrophilic matrix forming polymer; and
(d) from 0.2% to 5% lubricant.

2. The ophthalmic formulation of claim 1, wherein the combretastatin is combretastatin A4 phosphate or a pharmaceutically acceptable salt thereof.

3. The ophthalmic formulation of claim 1 comprising approximately 0.1% to 10% combretastatin A4 phosphate or a pharmaceutically acceptable salt thereof.

4. The ophthalmic formulation of claim 1, wherein the combretastatin is a compound of Formula I: 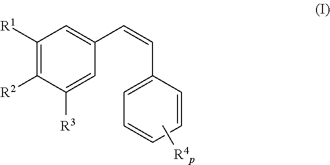
or a pharmaceutically acceptable salt thereof, wherein
each of R¹, R² and R³, independently of the others, is selected from the group consisting of hydrogen, C₁₋₆ alkoxy, and halogen, wherein at least two of R¹, R² and R³ are non-hydrogen;
R⁴ is selected from the group consisting of R⁵, R⁶, R⁵ substituted with one or more of the same or different R⁷ or R⁶, —OR⁷ substituted with one or more of the same or R⁷ or R⁶, —B(OR⁷)₂, —B(NR⁸R⁸)₂, —(CHR⁷)ₘ—R⁶, —S—(CH₂)ₘ—R⁶, —O—CHR⁷R⁶, —O—CR⁷(R⁶)₂, —O—(CHR⁷)ₘ—R⁶, —O— (CH₂)ₘ—CH[(CH₂)ₘR⁶]R⁶, —S—(CHR⁷)ₘ—R⁶, —C(O)NH—(CH₂)ₘ—R⁶, —C(O)NH—(CHR⁷)ₘ—R⁶, —O—(CH₂)ₘ—C(O)NH—(CH₂)ₘ—R⁶, —S—(CH₂)ₘ—C(O)NH—(CH₂)ₘ—R⁶, —O—(CHR⁷)ₘ—C(O)NH—(CHR⁷)ₘ—R⁶, —S—(CHR⁷)ₘ—C(O)NH—(CHR⁷)ₘ—R⁶, —NH—(CH₂)ₘ—R⁶, —NH—(CHR⁷)ₘ—R⁶, —N[(CH₂)ₘR⁶]₂, —NH—C(O)—NH—(CH₂)ₘ—R⁶, —NH—C(O)—(CH₂)ₘ—CHR⁶R⁶ and —NH—(CH₂)ₘ—C(O)—NH—(CH₂)ₘ—R⁶;
each R⁵ is independently selected from the group consisting of C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate
each R⁶ is a suitable group independently selected from the group consisting of ═O, —OR⁷, C₁₋₃ haloalkyloxy, —OCF₃, ═S, —SR⁷, ═NR⁷, ═NOR⁷, —NR⁸R⁸, halogen, —CF₃, —CN, —NC, —OCN, —SCN, —NO, —NO₂, ═N₂, —N₃, —S(O)R⁷, —S(O)₂R⁷, —S(O)₂OR⁷, —S(O)NR⁸R⁸, —S(O)₂NR⁸R⁸, —OS(O)R⁷, —OS(O)₂R⁷, —OS(O)₂OR⁷, —OS(O)₂NR⁸R⁸, —C(O)R⁷, —C(O)OR⁷, —C(O)NR⁸R⁸, —C(NH)NR⁸R⁸, —C(NR⁷)NR⁸R⁸, —C(NOH)R⁷, —C(NOH)NR⁸R⁸, —OC(O)R⁷, —OC(O)OR⁷, —OC(O)NR⁸R⁸, —OC(NH)NR⁸R⁸, —OC(NR⁷)NR⁸R⁸, —[NHC(O)]ₙR⁷, —[NR⁷C(O)]ₙR⁷, —[NHC(O)]ₙOR⁷, —[NR⁷C(O)]ₙOR⁷, —[NHC(O)]ₙNR⁸R⁸, —[NR⁷C(O)]ₙNR⁸R⁸, —[NHC(NH)]ₙNR⁸R⁸ and —[NR⁷C(NR⁷)]ₙNR⁸R⁸;
each R⁷ is independently selected from the group consisting of hydrogen, C₁₋₆ alkyl, C₃₋₈ cycloalkyl, C₄₋₁₁ cycloalkylalkyl, C₅₋₁₀ aryl, C₆₋₁₆ arylalkyl, 2-6 membered heteroalkyl, 3-8 membered cycloheteroalkyl, 4-11 membered cycloheteroalkylalkyl, 5-10 membered heteroaryl, 6-16 membered heteroarylalkyl, phosphate, phosphate ester, phosphonate, phosphorodiamidate, phosphoramidate monoester, phosphoramidate diester, cyclic phosphoramidate, cyclic phosphorodiamidate, and phosphonamidate;
each R⁸ is independently R⁷ or, alternatively, two R⁸ are taken together with the nitrogen atom to which they are bonded to form a 5 to 8-membered cycloheteroalkyl or heteroaryl which may optionally include one or more of the same or different additional heteroatoms and which may optionally be substituted with one or more of the same or different R⁷ or suitable R⁶ groups;
each m independently is an integer from 1 to 3;
each n independently is an integer from 0 to 3;
p is an integer from 1 to 5, and
wherein two adjacent R⁴ groups and their intervening atoms can be bonded to form a 5-8 membered ring fused to the central phenyl group.

5. The ophthalmic formulation of claim 1, wherein the combretastatin is a compound of Formula II: 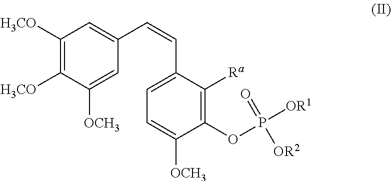
wherein
R^{a} is H or OP(O)(OR³)OR⁴; and
OR¹, OR², OR³ and OR⁴ are each, independently, OH, —O⁻QH⁺ or —O⁻M⁺, wherein M⁺ is a monovalent or divalent metal cation and Q is, independently:
a) an amino acid containing at least two nitrogen atoms where one of the nitrogen atoms, together with a proton, forms a quaternary ammonium cation QH⁺; or
b) an organic amine containing at least one nitrogen atom which, together with a proton, forms a quaternary ammonium cation, QH⁺.

6. The ophthalmic formulation of claim 5, wherein R^{a} is H, one of OR¹ and OR² is hydroxyl, and the other is —O⁻QH⁺ where Q is tris(hydroxymethyl)amino methane.

7. the ophthalmic formulation of claim 4, wherein the combretastatin is a compound of Formula III: 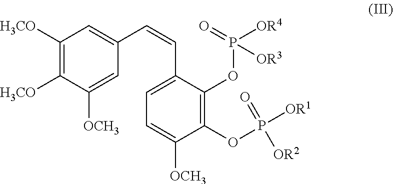
wherein
OR¹, OR², OR³ and OR⁴ are each, independently, OH, —O⁻QH⁺ or —O⁻M⁺, wherein M⁺ is a monovalent or divalent metal cation, and Q is, independently:
a) an amino acid containing at least two nitrogen atoms where one of the nitrogen atoms, together with a proton, forms a quaternary ammonium cation QH⁺; or
b) an organic containing at least one nitrogen atom which, together with a proton, forms a quaternary ammonium cation, QH⁺.

8. The ophthalmic formulation of claim 7, wherein at least one of OR¹, OR², OR³ and OR⁴ is hydroxyl, and at least one of OR¹, OR², OR³ and OR⁴ is —O⁻QH⁺, where Q is tromethamine.

9. The ophthalmic formulation of claim 1, further comprising an additional active agent.

10. The ophthalmic formulation of claim 9, wherein the additional active agent is selected from the group consisting of verteporfin, an inhibitor of Vascular Endothelial Growth Factor (VEGF), a VEGF trap molecule, and an antibody or fragment thereof directed to VEGF.

11. The ophthalmic formulation of claim 9, wherein the additional active agent is selected from the group consisting of a analgesic, an anesthetic, an anti-inflammatory agent, an antibiotic, an antifungal, an anti-allergic, an antiprotozoal agent, an antiviral agent, an antifungal agent, an anti-infective agent, an antimetabolite, and an antiangiogenic agent.

12. The ophthalmic formulation of claim 1, wherein the hydrophilic matrix forming polymer is selected from the group consisting of polyacrylic acid (carbomer), hydroxyethyl cellulose, hydroxypropyl cellulose, hydroxypropyl methylcellulose, carboxymethylcellulose, polyvinyl alcohol (PVA), alginic acids and pharmaceutically acceptable salts and/or mixtures thereof.

13. The ophthalmic formulation of claim 1, wherein the lubricant is selected from the group consisting of stearic acid, glyceryl behenate, magnesium stearate, calcium stearate, light mineral oil, polyethylene glycol, sodium stearyl fumarate, and hydrogenated vegetable oil.

14. An ocular bioadhesive tablet comprising
(a) a pharmaceutically effective amount of a combretastatin;
(b) from 60% to 95% w/w pre-gelatinized starch;
(c) from 1% to 10% w/w hydrophilic matrix forming polymer; and
(d) from 0.2% to 5% lubricant.

15. The ocular bioadhesive tablet of claim 14, wherein the tablet comprises approximately 0.1% to 10% w/w combretastatin.

16. The ocular bioadhesive tablet of claim 14, wherein the combretastatin is combretastatin A4 phosphate or an pharmaceutically acceptable salt thereof.

17. The ocular bioadhesive tablet of claim 16, wherein the pharmaceutically effective amount is 0.01 mg to 1.0 mg combretastatin A4 phosphate tromethamine.

18. The ocular bioadhesive tablet of claim 14, wherein the tablet weighs between approximately 5 mg and approximately 15 mg.

19. A method of treating an ocular vascular disease, said method comprising administering to a mammal in need thereof an ophthalmic formulation for ocular administration comprising:
(a) a pharmaceutically effective amount of a combretastatin;
(b) from 60% to 95% w/w pre-gelatinized starch;
(c) from 1% to 10% w/w hydrophilic matrix forming polymer; and
(d) from 0.2% to 5% lubricant.

20. The method of claim 19, wherein the ocular vascular disease is selected from the group consisting of a proliferative retinopathy, choroidal neovascularization (CNV), macular degeneration, diabetic retinopathy, ischemia-related retinopathy, diabetic macular edema, cystoid macular edema, pathological myopia, von Hippl-Landau disease, pathological choroidal vasculopathy (PCV), histoplasmosis of the eye, central retinal vein occlusion (CRVO), corneal neovascularization, retinal neovascularization, neovascular glaucoma, retinopathy of prematurity, vascularization of the cornea secondary to injury, retinitis pigmentosa (RP), uveal melanoma, retinoblastoma, choroidal melanoma, intraocular melanoma, and primary ocular lymphoma.
